(19) United States
(12) Patent Application Publication
(10) Pub. No.: US 2017/0340225 A1
(43) Pub. Date: Nov. 30, 2017

(54) SENSOR SYSTEM, IN PARTICULAR FOR DETERMINING A GLUCOSE CONCENTRATION

(71) Applicant: BIOTRONIK SE & Co. KG, Berlin (DE)

(72) Inventors: Jens Ulmer, Kuesnacht (CH); Ingo Lummer, Winterhur (CH)

(21) Appl. No.: 15/607,716

(22) Filed: May 30, 2017

(30) Foreign Application Priority Data
May 30, 2016 (DE) 10 2016 109 926.0

(51) Int. Cl. A61B 5/04; A61B 5/00; A61B 5/145; A61B 5/1468

(52) U.S. Cl. CPC A61B 5/04005; A61B 5/1468; A61B 5/6846; A61B 5/0031; A61B 5/14532; A61B 5/14; A61B 2562/0223

(57) ABSTRACT

A medical sensor system (1) for determining a feature in a human or animal body includes magnetic measurement nanoparticles (10) configured to form reversible chemical bonds with a binding substance, and experience a change in their magnetic relaxation behavior dependent on the formation of such bonds. The sensor system (i) further includes magnetic reference nanoparticles (20) having lesser (and preferably no) binding affinity to the binding substance.

### FIELD OF THE INVENTION

The present invention relates to sensor systems and methods for determining a feature in a human or animal body, in particular the concentration of an analyte such as glucose.

### BACKGROUND OF THE INVENTION

Medical sensor systems are known wherein at least parts of the system are inserted or implanted directly in a patient's body to capture physiological conditions, particularly concentrations of certain analytes, as precisely and directly as possible.

U.S. Pat. No. 9,538,942 (also European Patent EP 2 433 562 B1) describes a medical sensor system for detecting a feature in a human or animal body, including a signal pick-up unit which can be implanted into the body, and a signal processing unit spatially separated from the signal pick-up unit and having a transmitter and a receiver. The transmitter is designed to emit an alternating magnetic field to act on the signal pick-up unit, and the receiver is designed to receive a response signal of the signal pick-up unit, which can be generated by the magnetic interaction of the alternating magnetic field with the signal pick-up unit. The response signal is formed by a relaxation signal of a nanoparticle, and is caused by a change in the binding behavior between the nanoparticle and a further component of the sensor system, wherein the binding behavior is dependent on a concentration of an analyte to be determined.

However, in some circumstances, the determination of the analyte concentration may be inaccurate owing to external factors unrelated to the analyte. The system described in U.S. Pat. No. 9,538,942, for example, is susceptible with respect to the local particle concentration at the measuring site; the relative positions of (in particular the distances between) the nanoparticles and the measuring sensor; the viscosity at the measuring site; and the temperature. These parameters undergo continuous change, and cause drift in the sensor characteristic curve, requiring sensor recalibration.

### SUMMARY OF THE INVENTION

The present invention, which is defined by the claims set forth at the end of this document, seeks to provide sensor systems and methods which address the aforementioned problems.

An exemplary version of the invention involves a medical sensor system for determining a feature of interest (e.g., the concentration of an analyte) in a human or animal body, the system including magnetic measurement nanoparticles designed to form reversible chemical bonds with a binding substance (e.g., a receptor), wherein these bonds result in a change in the magnetic relaxation behavior of the measurement nanoparticles in an alternating magnetic field. The sensor system further includes magnetic reference nanoparticles which do not form the chemical bonds (or form them to a lesser degree), so that ideally their magnetic relaxation behavior is independent of the feature of interest, or at least is less dependent on the feature of interest than the magnetic relaxation behavior of the magnetic measurement nanoparticles. This allows use of magnetic susceptometry values of the reference nanoparticles to factor out interferences, such as those caused by temperature drift.

When it is stated that the reference nanoparticles form the chemical bond to a lesser degree, this means that the reference nanoparticles have a lower binding affinity (which may be characterized, for example, by the dissociation constant K_{D}) with respect to the binding substance. This binding affinity of the reference nanoparticles is preferably less than 10% of the binding affinity of the measurement nanoparticles with respect to the binding substance.

The reference nanoparticles thus form a reference system that, like the measurement nanoparticles, is exposed to the environmental/measuring conditions, but does not participate in the detection of the feature (or does so only to a small degree). Interferences can therefore be advantageously factored out, for example by forming ratios of measured values (such as relaxation signals).

A “nanoparticle” should be understood to mean a particle having a dimension (e.g., diameter) in the range of 1 nm to 500 nm. For reasons discussed below, the diameter of the nanoparticle is preferably greater than 11.2 nm.

The measurement and/or reference nanoparticles include a magnetic substance, such as compounds made of iron, nickel, cobalt, chromium and/or alloys made of manganese. These may include aluminum, copper, tin, antimony, arsenic, bismuth, boron or any other alloy, compound, or substance deemed expedient by a person skilled in the art.

The sensor system has measurement nanoparticles and reference nanoparticles having a mass concentration (mass per unit volume of sensor unit or signal pick-up unit) between 0.01 and 100 mg/ml, preferably between 0.1 and 50 mg/ml, with 1 to 10 m/gml being particularly preferred. A mass concentration of 1 mg/ml, for example, corresponds to a particle count of 3.3×10¹⁵, assuming a particle diameter of 20 nm.

The measurement/reference nanoparticle preferably includes Fe₃O₄, or is made of monodisperse iron oxide, whereby the nanoparticle is particularly versatile and is biocompatible.

The feature of interest to be determined by the invention is preferably the concentration of an analyte, in particular glucose. The magnetic measurement nanoparticles are each designed to fault reversible chemical bonds with a receptor or other binding substance in the presence of the analyte as a function of the concentration of the analyte, wherein these bonds result in the change in the magnetic relaxation behavior of the measurement nanoparticle.

An “analyte” should be understood to mean the molecule or the substance which is to be detected by the sensor system, or which is to have its concentration determined, as a feature of interest in the human and/or animal body. The analyte may be a sugar or a sugar compound (such as glucose, sucrose, fructose, lactose, maltose, dextrose and/or glycogen), a protein, a salt or a mineral (such as calcium, phosphate, potassium, sulfur, sodium, chloride, magnesium, iron, iodide, manganese, copper, cobalt, zinc, fluoride, selenium and/or chromium), an ion (such as hydrogen, sodium, potassium, ammonium, hydronium, magnesium, calcium, iron II, iron III, fluoride, chloride, iodide, hydroxyl, nitrate, bicarbonate, oxide, sulfate and/or phosphate), and/or another substance.

Apart from analyte (e.g., glucose) concentration, a “feature” can be understood to mean a parameter of a substance such as a pH value, a charge (for example of an ion), a temperature, a size, a mass, a state of matter, and a presence or an absence (and/or a quantity) of a substance. A feature could alternatively or additionally be any other feature that a person skilled in the art deems useful, as long as the presence of the feature influences the relaxation behavior of the measurement nanoparticles. The feature preferably relates to a variable component of the human and/or animal body.

The aforementioned “binding substance” is preferably a receptor for the analyte. A “receptor” should be understood to mean a molecule or other substance that specifically binds the analyte and, given the similarity of the analog (discussed below) with the analyte, also specifically binds the analog. The receptor may be a peptide, a protein (including antibodies and fragments thereof, RNA, or DNA), aptamers and the relatives thereof, cyclic macromolecules (including selectively binding ions or ionophores), non-cyclic macromolecules (including polymers having identifying features impressed during polymerization, i.e., molecular imprinted polymers), or other materials suitable for use as receptors.

The term “reversible” refers to the ability to be reversed or detachable.

In a preferred version of the sensor system, the measurement nanoparticles each include a magnetic core, wherein the cores are each coated with an analog of the analyte, so that the measurement nanoparticles (including the analog) reversibly chemically bind to the receptor of the analyte and the analog as a function of the concentration of the analyte. Preferably, the magnetic measurement nanoparticles (including the analog) are reversibly bound to the receptor of the analyte and the analog by way of physisorption, and as a function of the concentration of the analyte.

An “analog” should be understood to mean a molecule and/or a substance that is bindable to or bound by the same receptor as the analyte, due to similarity in structure, charge distribution, and/or another characteristic,

The analog is preferably a dextran, and/or the receptor is preferably concanavalin A (“ConA”). The dextran preferably has a molecular weight between 40,000 Da and 200,000 Da.

It is also possible for the cores of the measurement nanoparticles to be coated with the receptor of the analyte. The sensor system or the signal pick-up unit then includes the analog (i.e., here the binding substance is the analog of the analyte). A displacement reaction thus takes place, in which the analyte displaces the analog from the receptor if it is present in an appropriate concentration.

The signal pick-up unit (discussed below) thus includes the receptor where the measurement nanoparticle is coated with the analog, and includes the analog where the measurement nanoparticle is coated with the receptor. To this end, they are preferably disposed on a fixed structure, such as a mesh or lattice structure. The presence of these antagonists results in reversible binding between the analog and the receptor.

Analyte detection is based on a displacement reaction wherein the analyte displaces the analog from the receptor (where cores are coated with the receptor), or on competitive binding of the analyte to the receptor (where cores are coated with the analog). If the local concentration of the analyte in the analyzed tissue changes, the number of binding sites between the analog and the receptor changes, and a freedom of movement or rotation (in particular a magnetic relaxation) of the functionalized magnetic measurement nanoparticle changes. This can be measured in an alternating magnetic field, for example in a field having a magnetic flux density with an amplitude of 0.5 mT. An “alternating magnetic field” should be understood to mean a magnetic field having changing intensity and/or polarity (caused, for example, by alternating voltage or current).

The signal pick-up unit (discussed below) is preferably designed as a sensor for a glucose analyte.

The magnetic reference nanoparticles each include a magnetic core, which is preferably coated with polyethylene glycol (PEG), in particular with PEG300.

In contrast to the measurement nanoparticles, the reference nanoparticles are not coated with the analog/receptor or otherwise functionalized, and therefore they do not participate (or only minimally participate) in the detection reaction. The relaxation behavior of the reference nanoparticles changes only in accordance with general ambient conditions, whereby they serve as the reference system.

The sensor system preferably includes a signal pick-up unit which can be implanted into a human and/or animal body and which includes the measurement nanoparticles, reference nanoparticles, and the receptor or other binding substance. A signal processing unit is spatially separated from the signal pick-up unit, preferably ex vivo, and includes a transmitter and a receiver. The transmitter is designed to emit an alternating magnetic field to act on the measurement nanoparticles and reference nanoparticles of the signal pick-up unit. The receiver is designed to receive a response signal in the form of a relaxation signal of the signal pick-up unit, which can be generated by the magnetic interaction of the alternating magnetic field with the magnetic measurement nanoparticles and the magnetic reference nanoparticles of the signal pick-up unit.

The sensor system preferably includes a calculation unit which assists in calculating the concentration of the analyte, e.g., a microprocessor, application-specific integrated circuit, or similar device. The calculation unit is preferably connected to the signal processing unit, and is designed to calculate from the response signal at least the imaginary part of the dynamic susceptibility of the magnetic measurement nanoparticles and of the reference nanoparticle for frequencies of the alternating magnetic field. To calculate the concentration of the analyte, the calculation unit is preferably configured to determine a relationship (preferably a ratio) between the amplitude of a first peak corresponding to the imaginary part of the dynamic susceptibility assigned to the reference nanoparticles, and the amplitude of a second peak corresponding to the imaginary part of the dynamic susceptibility assigned to the measurement nanoparticles. (The “imaginary part of the dynamic susceptibility” refers to the mathematical value of such an imaginary part)

The particle size or the diameter of the measurement nanoparticle or reference nanoparticle is selected such that the Neel relaxation is negligible. This is the case when the time scale of the measurements is shorter than the Neel relaxation times. Thus, measurement nanoparticles or reference nanoparticles having diameters greater than 11.2 nm are preferred.

A preferred version of the sensor system includes a hydrogel, wherein the measurement nanoparticles, the reference nanoparticles, and the receptor or other binding substance are disposed in the hydrogel. The binding substance can be bound to the hydrogel. If the measurement nanoparticles include the receptor in the form of a coating, the analog is preferably provided in the hydrogel or bound thereto.

A hydrogel is a gel made of a polymer or copolymer which contains water, but is water-insoluble. Furthermore, the molecules of the hydrogel are linked chemically, for example by covalent or ionic bonds, or physically, such as by looping polymer chains, to form a three-dimensional network. The reader is referred to U.S. Pat. No. 9,538,942 for possible hydrogels that might be used with the invention. (The contents of U.S. Pat. No. 9,538,942 are hereby incorporated by reference, such that its contents should be regarded as a part of this document's contents.)

The hydrogel preferably has a consistency that allows the hydrogel to be easily subcutaneously injected. The hydrogel is preferably suspended in a solution suitable for injection purposes as a dispersion of gel spherules measuring 200 to 300 micrometers. However, the hydrogel may gel in situ, for example due to a temperature change.

The signal pick-up unit or the biosensor can therefore be easily injected into tissue, and no or very few foreign body reactions occur after implantation. Because there are few or no foreign body reactions or inflammatory reactions, conditions at the implantation site remain unaffected, thereby creating optimal detection conditions.

The signal pick-up unit is at preferably fully (or at least partially) biodegradable, that is, capable of disintegrating in the human/animal body. The measures described in U.S. Pat. No. 9,538,942 can be used for this purpose.

The invention also involves a method for determining a feature (e.g., the concentration of an analyte such as glucose) in a human or animal body using a medical sensor system as described in this document. An alternating magnetic field is emitted to act on the measurement nanoparticles and reference nanoparticles, and a response signal (relaxation signal) is received. The response signal is generated by magnetic interaction of the alternating magnetic field with the measurement nanoparticles and the reference nanoparticles. The concentration of the analyte, or the other feature of interest, is determined or detected via the received relaxation signal. While this method assumes that the signal pick-up unit has been implanted in a human or animal body, the actual implantation step(s) are not regarded to be an essential part of the invention.

Preferably, when calculating the concentration of the analyte, at least the imaginary part of the dynamic susceptibility of the magnetic measurement nanoparticles and of the reference nanoparticles is calculated with the aid of the response signal for various frequencies of the alternating magnetic field. To calculate the concentration of the analyte, a relationship preferably a ratio—is determined between the amplitude of a first peak corresponding to the imaginary part of the dynamic susceptibility assigned to the reference nanoparticles, and the amplitude of a second peak corresponding to the imaginary part of the dynamic susceptibility assigned to the measurement nanoparticles.

Further advantages, features, and objects of the invention will be apparent from the remainder of this document in conjunction with the associated drawings.

### BRIEF DESCRIPTION OF THE DRAWINGS

FIG. 1 schematically illustrates an exemplary sensor system according to the invention.

FIG. 2 schematically illustrates a competitive binding system usable in the invention.

FIG. 3 shows the measured imaginary part of the dynamic susceptibility of the measurement nanoparticles and reference particles plotted against the frequency of the exciting alternating magnetic field for various concentrations of the analyte (here glucose).

FIG. 4 shows the amplitude ratio A₁/A₂ of the peaks of the imaginary part of the dynamic susceptibility for various analyte (glucose) concentrations.

### DETAILED DESCRIPTION OF EXEMPLARY VERSIONS OF THE INVENTION

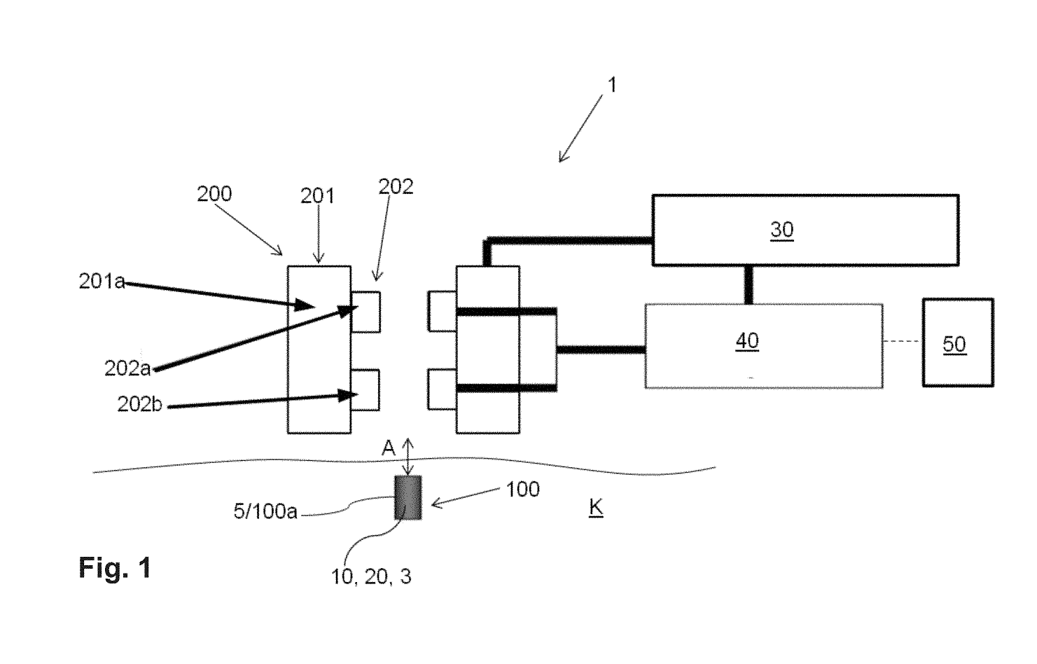

FIG. 1 schematically illustrates a medical sensor system 1 for determining a feature, such as the concentration of an analyte, in a human or animal body. The following discussion will assume glucose is the analyte in question, and that glucose concentration is the feature being measured, but other analytes and features could be measured instead.

20

In FIG. 1, the sensor system 1 includes a signal pick-up unit 100 including magnetic measurement nanoparticles 10, whose magnetic relaxation behavior changes when they form reversible chemical bonds with a binding substance 3 (here a receptor for glucose), and in particular concanavalin A (“ConA”).

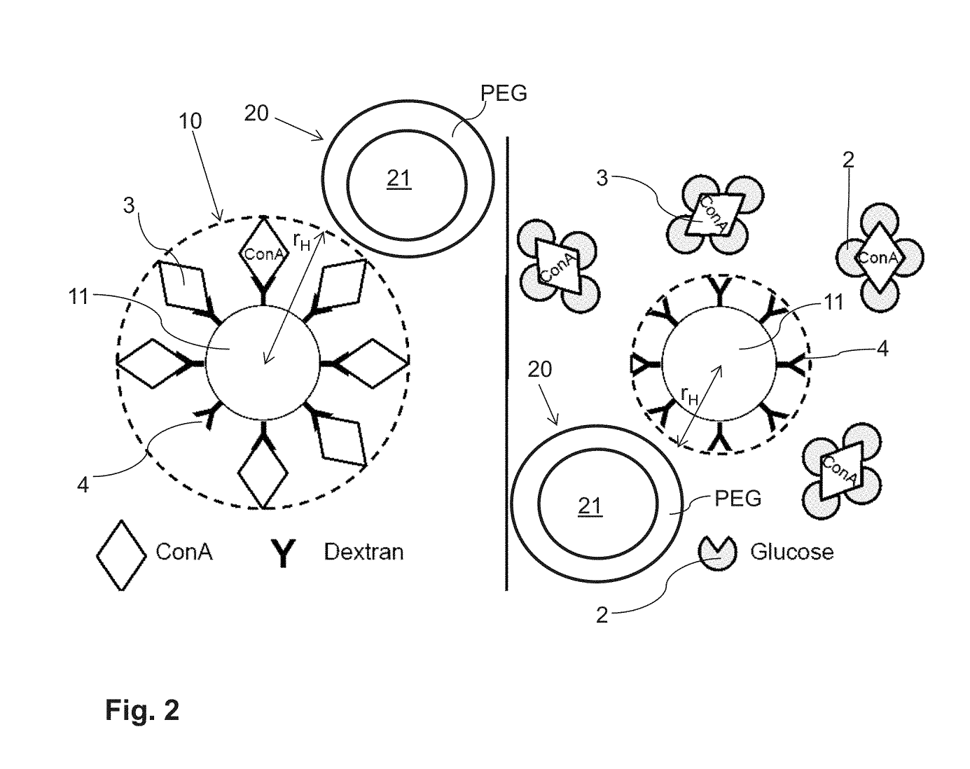

As seen in FIG. 2, a competing binding system can be used wherein the magnetic cores 11 of the measurement nanoparticles 10 are coated with an analog 4 of the (glucose) analyte 2, e.g., dextran. The dextran analog 4 preferably has a molecular weight between 40,000 Da and 200,000 Da.

If the concentration of the glucose analyte 2 is low in the region of the signal pick-up unit 100, the ConA receptors (or other binding substance) 3 reversibly bind(s) to a greater degree to the dextran analog 4, as seen at the left side of FIG. 2. As a result, the hydrodynamic radius of the measurement nanoparticles 10 increases, which impacts the Brown relaxation. At a comparatively high glucose analyte 2 concentration, the glucose analyte 2 reversibly bind(s) to the ConA receptors 3, and the hydrodynamic radius r_{H} of the measurement nanoparticles 10 accordingly decreases, as seen at the right side of FIG. 2

The signal pick-up unit 100 also includes magnetic reference nanoparticles 20, which do not form the chemical bond with the ConA receptors 3, or at least form this bond to a lesser degree than the glucose analyte 2 and/or the dextran analog 4. The reference nanoparticles 20 may be, for example, magnetic cores coated with PEG, preferably PEG300 (i.e., PEG having a chain length of 300 monomers).

FIG. 1 schematically depicts the measurement nanoparticles 10, reference nanoparticles 20, and the receptor 3 provided in a hydrogel 5, with these components 10, 20, 3, and 5 collectively providing the signal pick-up unit 100, which can be easily implanted or injected into the human or animal body K. The analyte 2 within the body can then diffuse into the hydrogel 5 of the signal pick-up unit 100, so that the reactions depicted in FIG. 2 can occur in the signal pick-up unit 100.

In an alternative version, the signal pick-up unit 100 includes a surrounding membrane or other permeable shell 100a, which permanently encloses the measurement nanoparticles 10, reference nanoparticles 20, and the receptor 3, and which can be penetrated by the glucose or other analyte 2. In this case, the analyte 2 diffuses from the surroundings through the shell 100a and into the :interior of the signal pick-up unit 100 implanted in the body K, so that the reactions depicted in FIG. 2 can occur in the signal pick-up unit 100.

In addition to the signal pick-up unit 100, the sensor system 1 preferably also includes a signal processing unit 200 (FIG. 1), which can be disposed spatially separated from the signal pick-up unit 100, in particular outside the body K. The signal processing unit 200 includes a transmitter 201 and a receiver 202, wherein the transmitter 201 is designed to emit an alternating magnetic field to act on the magnetic measurement nanoparticles 10 and reference nanoparticles 20 of the signal pick-up unit 100. The receiver 202 is designed to receive a response signal in the form of a relaxation signal of the signal pick-up unit 100, which can be generated by the magnetic interaction of the alternating magnetic field with the magnetic measurement nanoparticles 10 and the magnetic reference nanoparticles 20 of the signal pick-up unit 100. For generating the alternating magnetic field, the transmitter 201 can include a transmitting coil 201a, which can be suitably connected to an AC voltage source 30. The receiver 202 can further include two receiving coils 202a, 202b for receiving the magnetic relaxation signals, wherein these receiving coils 202a, 202b can be connected in a known manner to a lock-in amplifier 40 for evaluating the relaxation signals. The amplifier 40 multiplies the signals of the receiving coils 202a, 202b with the la excitation frequency 30 of the AC voltage signal.

Referring to FIG. 1, the distance A between the signal pick-up unit 100 and the transmitter 201 (particularly the transmitting coil 201a) can he, for example, in the range of 1 to 10 mm, preferably within 1-2 mm of 5 mm. The generated alternating magnetic field can have an amplitude in the range of (for example) 0 to 600 μT.

The diameters/dimensions of the nanoparticles 10, 20 are preferably selected such that the Neel relaxation time at a body temperature of 37° C. is greater by three orders of magnitude than the Brown relaxation time. This is typically achievable if the diameters of the nanoparticles 10, 20 are greater than approximately 11.2 nm.

In an exemplary version of the invention, the measurement nanoparticles 10 include a core 11 made of ferromagnetic elements such as iron, cobalt, nickel, and/or of a chemical compound of these elements. The core 11 is coated with a dextran analog 4, wherein the diameter of the resulting nanoparticle 10 is on average approximately 80 nm. The dextran preferably has a molecular weight between 40,000 Da and 200,000 Da. The reference nanoparticles 20 have a core 21 made of iron oxide, which is coated with PEG300, thereby providing coated reference nanoparticles having an average diameter of approximately 130 nm.

The dynamic susceptibility of the measurement or reference nanoparticles 10, 20 for the Brown relaxation present here is:

$$χ = \frac{{χ}_{0}}{1 + i {ωτ}_{B}}$$

$$wherein$$

$${τ}_{B} = \frac{4 π {r}_{H}^{3} η}{{k}_{B} T}$$

is the Brown relaxation time. The particle 10, 20 rotates mechanically against the viscous forces of the surrounding medium. The Brown relaxation time is proportional to the third power of what is known as the hydrodynamic radius r_{H} of the particles, which is plotted in FIG. 2, and which is decisively influenced by the binding of the measurement nanoparticles 10 to the receptor 3. In the case of a low glucose analyte 2 concentration, an accordingly high number of ConA receptor molecules 3 bind to the measurement nanoparticle 10, whereby its hydrodynamic radius r_{H} increases (schematically depicted at the left side of FIG. 2). In contrast, at a high glucose concentration, the hydrodynamic radius r_{H} decreases, since now the ConA receptors 3 are bound to a greater extent by the glucose analyte 2.

The foregoing competitive binding can be evaluated by measuring the magnetic relaxation behavior of the measurement nanoparticles 10 or reference nanoparticles 20. For this purpose, the receiver 202 preferably includes two receiving coils 202a, 202b disposed coaxially with one another (as seen in FIG. 1). The alternating magnetic field induces a voltage V_{Coil1}, V_{Coil2} in the receiving coils 202a, 202b, whereby the difference of these voltages Vₘ=V_{Coil1}−V_{Coil2} can be measured

A voltage V_{Excitation} is induced in the receiving coils 202a, 202b of the receiver 202, with V_{Excitation} being dependent on the alternating magnetic field, as well as a voltage V_{Particles}, which stems from the magnetization of the measurement or reference nanoparticles 10, 20. As a result, V_{Coil}=V_{Excitation}−V_{Particles}.

Where the receiver coils 202a, 202b are disposed in the transmitter coil 201a, as in FIG. 1, the induced voltage V_{Excitation} is the same for the two coils 202a, 202b, so that Vₘ includes only the particle contributions. If the signal pick-up unit 100 is not present, Vₘ vanishes.

When a signal pick-up unit 100 is present, the lock-in amplifier 40 may be used to determine portions of the voltage Vₘ which are in phase with the alternating magnetic field (i.e., the real part of Vₘ), and which are phase shifted 90° thereto (i.e., the imaginary part of Vₘ). The imaginary part of the dynamic susceptibility thus results as

$$χ = C \cdot \frac{{V}_{m}}{ω}$$

wherein Vₘ is the imaginary part of the voltage Vₘ measured by way of the receiver coils 202a and 202b; ω is the frequency of the alternating magnetic field; and C is a constant.

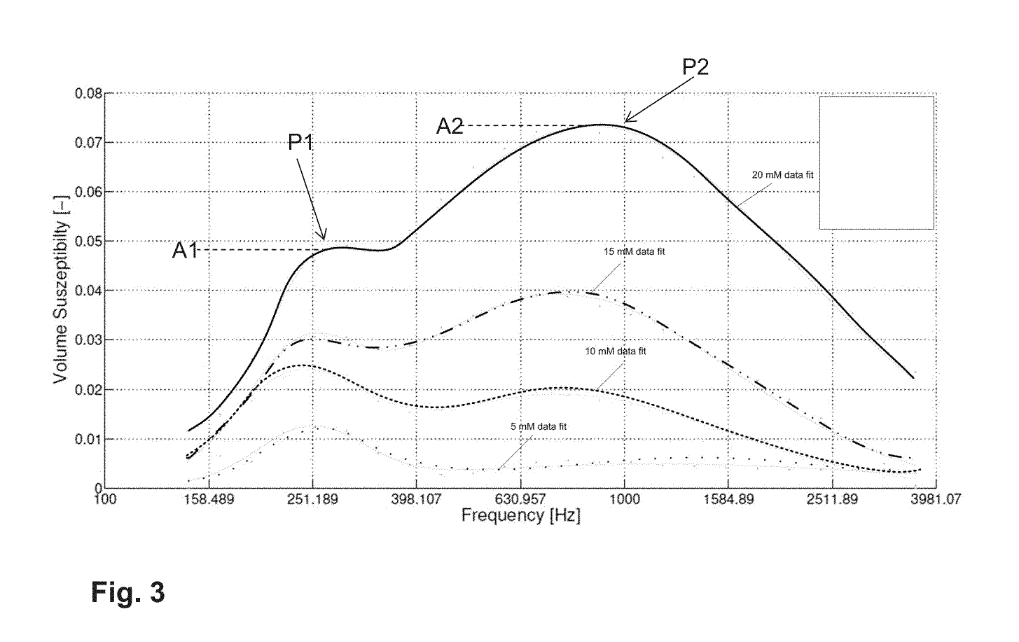

It has been found that long particle chains form in the binding system of FIG. 2 at low glucose concentrations, resulting in sedimentation of these chains. This sedimentation phenomenon has been found to be reversible, and is reflected in the measured amplitude of the imaginary part of the dynamic susceptibility, shown in FIG. 3 for the nanoparticles 10, 20 for various glucose concentrations (5 mM, 10 mM, 15 mM and 20 mM).

In detail, the following sample compositions were used:

| | Measurement | Reference | Molar |
|---|---|---|---|
| | nanoparticles 10 | nanoparticles 20 | ratio cₚ |
| Glucose | (80 nm dextran) | (130 nm PEG) | ConA/nano- |
| [mM] | [mg/ml] | [mg/ml] | particles |
| 0-20 | 6 | 4.8 | 165 |

The individual sample volumes used had the following compositions (components 80 nm dextran (25 mg/ml), 130 nm PEG (10 mg/ml), PBS (phosphate-buffered saline solution), and glucose (in PBS, 100 mM)):

| 80 nm | 130 nm | | | | Total |
|---|---|---|---|---|---|
| dextran | PEG | PBS | ConA | Glucose | volume |
| [μL] | [μL] | [μL] | [μL] | [μL] | [μL] |
| 4.80 | 9.60 | 4.00 | 1.60 | — | 20.00 |
| 4.80 | 9.60 | 3.00 | 1.60 | 1.00 | 20.00 |
| 4.80 | 9.60 | 2.00 | 1.60 | 2.00 | 20.00 |
| 4.80 | 9.60 | 1.00 | 1.60 | 3.00 | 20.00 |
| 4.80 | 9.60 | — | 1.60 | 4.00 | 20.00 |

However, the measured signal (or the amplitude of the imaginary part of the dynamic susceptibility) of FIG. 3 also includes information about the distance of the receiver 202 from the signal pick-up unit 100, the dilution of the particles, the number of the particles, and the viscosity, in addition to the sedimentation.

The reference system formed by the reference nanoparticles 20 is therefore used. FIG. 3 shows, for example, that two peaks P1, P2 can be observed at a glucose concentration of 20 mM, wherein a first peak P1 occurs at approximately 250 Hz (alternating field frequency) and the second peak P2 at approximately 800 Hz. These peaks P1, P2 correspond to the measurement nanoparticles 10 and reference nanoparticles 20, with the low frequency peak P1 being created by the 130 nM PEG particles 20, and the high frequency peak P2 being created by the 80 nm dextran particles 10. The plotted lines bearing the peaks P1 and P2 represent the overlapping relaxation signals of the two particle types 10, 20, with the is increases in amplitude arising when measurement particles 10 have lesser attachment between their dextran analog coatings 4 and the ConA receptor 3 (i.e., when concentration of the glucose analyte 2 is higher). Since the measurement nanoparticle peaks P2 rise considerably more strongly than the reference nanoparticle peaks P2, the ratios of the amplitudes A1, A2 may be considered for determining the glucose concentration:

$$\begin{matrix}y = {A}_{1} / {A}_{2} \\ = \frac{amplitude measured at 220 Hz in FIG . 3}{amplitude measured at 894 Hz in FIG . 3}\end{matrix}$$

The ratio can be evaluated in an automated fashion, e.g., by an calculation unit 50 (FIG. 1). Calculating the ratios at various glucose concentrations results in:

| Glucose [mM] | A₁/A₂ |
|---|---|
| 0 | 1.6048 |
| 5 | 1.1385 |
| 10 | 0.9910 |
| 15 | 0.9335 |
| 20 | 0.8920 |

With an appropriately calibrated sensor system 1, the ratio determination A₁/A₂ allows the glucose (or other analyte) concentration to be determined in the region of the signal pick-up unit 100, with the reference system reducing the effect of interferences.

The signal-to-noise ratio was determined by way of repeat measurements. The standard deviation of A₁/A₂ was determined to be 0.001377 (using 22 measurements),

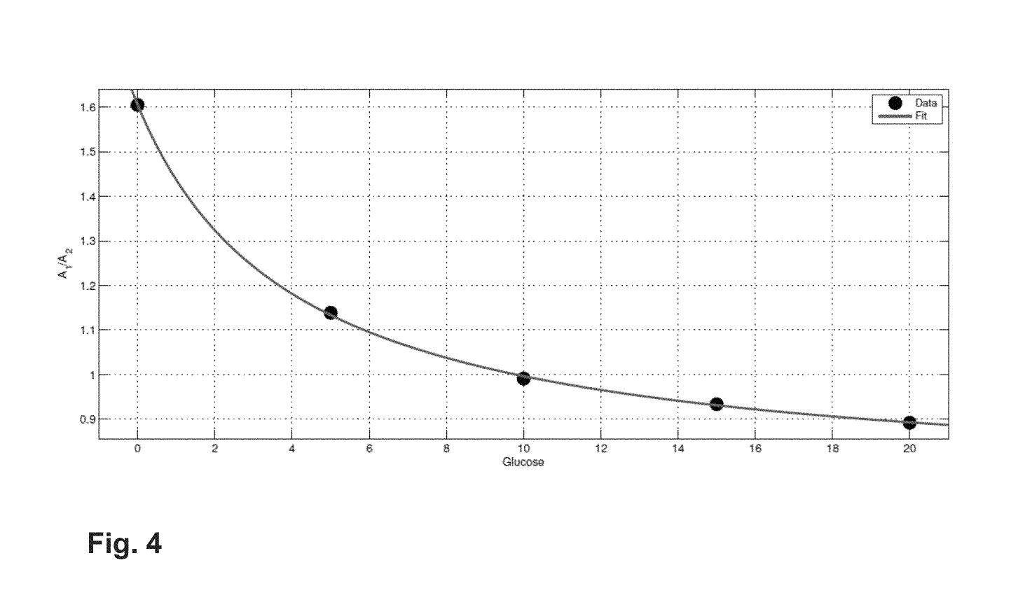

FIG. 4 shows the foregoing A₁/A₂ ratio plotted against the glucose concentration, along with a curve fit to the data. The A₁/A₂ ratio was fit to the following function:

$$\frac{{A}_{1}}{{A}_{2}} = \frac{aG + b}{G + d}$$

wherein G is the glucose concentration, and a, b, d are fit parameters. The illustrated curve fit has an R² value of 0.9998, showing good agreement with the foregoing function. The nonlinearity arises because the two particle types 10, 20 interact with ConA receptor 3 and thus agglomerate or sediment.

To obtain a sensor system 1 having linear characteristics, a reference nanoparticle 20 that does not bind to the receptor 3 can be used. It is also possible to determine the superimposition of the Brown relaxation of the two particle types 10, 20 through individual measurements of the two particles 10, 20, and subtract these from each other:

$$y = \frac{{A}_{2}}{{A}_{1} - a * {A}_{2}}$$

The factor a then includes the influence of the measurement nanoparticles 10 (80 nm dextran) on A₁ and the sedimentation of the reference nanoparticles 20 (130 nm PEG). Using the foregoing data, linear behavior is obtained for a=0.75.

The invention is not limited to the exemplary versions described above, and rather is limited only by the claims set out below. Thus, the invention encompasses all different versions that fall literally or equivalently within the scope of these claims.

What is claimed is:

1. A medical sensor system (1) for determining a feature in a body (K), the system including:
a. magnetic measurement nanoparticles (10) having magnetic relaxation behavior dependent on their formation of reversible chemical bonds with a binding substance (3); and
b. magnetic reference nanoparticles (20) which:
(1) do not form chemical bonds with the binding substance (3), or
(2) have a lower binding affinity to the binding substance (3) than the magnetic measurement nanoparticles (10).

2. The sensor system of claim 1 wherein:
a. the feature is the concentration of an analyte (2);
b. the magnetic measurement nanoparticles (10) are configured to form reversible chemical bonds with the binding substance (3) in the presence of the analyte (2) in dependence on the concentration of the analyte (2), the bonds resulting in changes in the magnetic relaxation behavior of the magnetic measurement nanoparticles (10).

3. The sensor system of claim 2 wherein the analyte (2) is glucose.

4. The sensor system of claim 2 wherein:
a. the binding substance (3) is a receptor for:
(1) the analyte (2), and
(2) an analog (4) of the analyte (2),
b. the magnetic measurement nanoparticles (10) each include a magnetic core (11), the cores (11) having the analog (4) thereon, whereby the magnetic measurement nanoparticles (10) reversibly chemically bind to the receptor (3) of the analyte (2) and the analog (4) in dependence on the concentration of the analyte (2).

5. The sensor system of claim 4 wherein:
a. the analog (4) is dextrin), and/or
b. the binding substance (3) is concanavalin A.

6. The sensor system of claim 2 wherein:
a. the binding substance (3) is a receptor for the analyte (2), and
b. the magnetic measurement nanoparticles (10) each include a magnetic core (11) coated with a receptor (3) of the analyte (2), whereby the magnetic measurement nanoparticles (10) reversibly chemically bind to the analog (4) in dependence on the concentration of the analyte (2).

7. The sensor system of claim 6 wherein:
a. the analog (4) is dextrin), and/or
b. the binding substance (3) is concanavalin A.

8. The sensor system of claim 7 wherein the analyte (2) is glucose.

9. The sensor system of claim 1 wherein the magnetic reference nanoparticles (20) each include a magnetic core (21) coated with polyethylene glycol.

10. The sensor system of claim 1 wherein the magnetic measurement nanoparticles (10) each bear an analog (4) of an analyte (2) thereon, wherein the analog (4) forms the reversible chemical bonds with the binding substance (3).

11. The sensor system of claim 10 wherein the feature is the concentration of the analyte (2).

12. The sensor system of claim t:
a. further including the binding substance (3),
b. wherein the measurement nanoparticles (10), reference nanoparticles (20), and the binding substance (3) are provided as a signal pick-up unit (100) configured for implantation into a human or animal body (K).

13. The sensor system of claim 12 further including a signal processing unit (200) spaced from the signal pick-up unit (100), the signal processing unit (200) including:
a. a transmitter (201) configured to emit an alternating magnetic field which magnetically interacts with the magnetic measurement nanoparticles (10) and the reference particles (20) of the signal pick-up unit (100),
b. a receiver (202) configured to receive a relaxation response signal from the signal pick-up unit (100), the relaxation response signal being dependent on the magnetic interaction with the magnetic measurement nanoparticles (10) and the reference particles (20) of the signal pick-up unit (100).

14. The sensor system of claim 13 wherein the signal processing unit (200) includes a calculation unit (50) configured to calculate:
a. the imaginary part of the dynamic susceptibility (χ) of:
(1) the magnetic measurement nanoparticles (10), and
(2) the reference nanoparticles (20),
from the relaxation response signal; and
b. a relationship between:
(1) the amplitude (A₁) of a peak (P1) in the imaginary part of the dynamic susceptibility (χ) of the reference nanoparticles (20), and
(2) the amplitude (A₂) of a peak (P2) in the imaginary part of the dynamic susceptibility (χ) of the measurement nanoparticles (10).

15. The sensor system of claim 12 wherein:
a. the signal pick-up unit (100) further includes a hydrogel (5), and
b. the measurement nanoparticles (10), the reference nanoparticles (20), and the binding substance (3) are disposed within the hydrogel (5).

16. The sensor system of claim 12 wherein:
a. the signal pick-up unit (100) further includes a permeable shell (100a), and
b. the measurement nanoparticles (10), the reference nanoparticles (20), and the binding substance (3) are disposed within the permeable shell (100a).

17. The sensor system of claim 12 wherein the signal pick-up unit (100) is at least partially biodegradable.

18. A method for determining a feature in a body (K) using the medical sensor system (1) of claim 1, the method including the steps of:
a. emitting an alternating magnetic field onto the measurement nanoparticles (10) and reference nanoparticles (20),
b. measuring a relaxation response signal, the relaxation response signal being dependent on the magnetic interaction of the alternating magnetic field with the magnetic measurement nanoparticles (10) and the reference particles (20).

19. The method of claim 20 further including the steps of calculating:
a. the imaginary part of the dynamic susceptibility (χ) of:
(1) the magnetic measurement nanoparticles (10), and
(2) the reference nanoparticles (20),
from the relaxation response signal; and
b. a relationship between:
(1) the amplitude (A₁) of a peak (P1) in the imaginary part of the dynamic susceptibility (χ) of the reference nanoparticles (20), and
(2) the amplitude (A₂) of a peak (P2) in the imaginary part of the dynamic susceptibility (χ) of the measurement nanoparticles (10).

20. The method if claim 19 wherein the calculated relationship is a ratio of the amplitudes (A₁, A₂).
